(19) United States
(12) Patent Application Publication
(10) Pub. No.: US 2022/0152262 A1
(43) Pub. Date: May 19, 2022

(54) Vortex Driven Ozone Distribution System

(71) Applicant: Quail Systems, LLC, Adamsville, TN (US)

(72) Inventor: Robert W. Smith, Adamsville, TN (US)

(73) Assignee: Quail Systems, LLC, Adamsville, TN (US)

(21) Appl. No.: 17/485,817

(22) Filed: Sep. 27, 2021

Related U.S. Application Data
(60) Provisional application No. 63/083,518, filed on Sep. 25, 2020.

(51) Int. Cl. A61L 9/12; A61L 2/20; A61L 2/24

(52) U.S. Cl. CPC A61L 9/122; A61L 2/202; A61L 2/24; A61L 2202/14; A61L 2209/212; A61L 2202/16; A61L 2202/25; A61L 2209/111; A61L 2209/134; A61L 2202/15

(57) ABSTRACT

A portable, vortex driven ozone distributor is disclosed that includes a vortex van and multiple ozone generation units within a canister. The vortex fan in combination with the canister provides the ability generate a high velocity stream of ozonated air that may be directed against the ceiling of a room. The high velocity of the ozonated airstream permits all surfaces of the room to be sanitized by the ozone because the air traverses the ceiling and follows the walls down to contact all surfaces of the room.

### REFERENCE TO RELATED APPLICATIONS

This application claims the benefit of U.S. Provisional Application No. 63/083,518 entitled “Vortex Driven Ozone Distribution System” filed Sep. 25, 2020, which is incorporated by reference in its entirety.

### BACKGROUND

The ability to sanitize an enclosed space such as a room quickly and efficiently is difficult. Conventional methods relying on chemical sterilants are an imperfect solution unless the room is equipped with a drain and has interior contents capable of being subjected to a liquid spray. However, even in rooms design for such spray sterilization, nooks and crannies remain that can harbor pathogens. Furthermore, there is no effective way to sanitize paper and other soft or absorptive materials that would be damaged by liquid contact.

UV lights and ozone are two ways to sanitize an enclosed space without liquids. Conventional UV light systems have the disadvantage that many lights are required and any shadowed area, such as the inside of a desk drawer or the area under a desk, are not sanitized as these areas are shadowed from the light. Conventional ozone systems suffer from at least one of insufficient ozone concentration generation per unit time and the inability to properly circulate the produced ozone throughout the room in an efficient manner. In either instance the primary disadvantage is that room sterilization cannot be accomplished quickly, thus in less than one hour. Furthermore, easy portability is often lacking from conventional UV light and ozone systems, making it difficult to sanitize multiple rooms successively.

As can be seen from the above description, there is an ongoing need for simple and efficient devices and methods to rapidly and efficiently sanitize rooms that are unsuitable for liquid spray sterilization.

### SUMMARY

In one aspect, the invention provides a portable, vortex driven ozone distributor that includes a lid connected to a canister that resides on a base, where the canister includes at least two ozone generation units fixed within the canister where the ozone generation units comprise ozone generation plates and energize circuitry; at least one control module in electrical communication with the at least two ozone generation units configured to instruct the energize circuitry to energize the ozone generation plates with a first applied electric potential, where the at least one control module can instruct one or more of the at least two ozone generation units in response to a control signal; and a vortex fan in electrical communication with the at least one control module, where the vortex fan comprises a vortex generating housing and fan blade configured to spin in response to a second applied electric potential provided in response to the at least one control module.

In another aspect of the invention, there is a method of using the vortex driven ozone distributor to sanitize multiple enclosed airspaces, such as multiple rooms of a building, the method including placing at least two of the distributors in at least two enclosed airspaces; activating the distributors to sanitize the at least two enclosed airspaces; moving the at least two distributors to at least two different enclosed airspaces; and activating the distributors to sanitize the at least two different enclosed airspaces.

Other systems, methods, features and advantages of the invention will be, or will become, apparent to one with skill in the art upon examination of the following figures and detailed description. It is intended that all such additional systems, methods, features, and advantages be included within this description, be within the scope of the invention, and be protected by the claims that follow. The scope of the present invention is defined solely by the appended claims and is not affected by the statements within this summary.

### BRIEF DESCRIPTION OF THE FIGURES

The invention can be better understood with reference to the following drawings and description. The components in the figures are not necessarily to scale, emphasis instead being placed upon illustrating the principles of the invention.

FIG. 1 represents a portable, vortex driven ozone distributor.

FIG. 2 represents a top view of an ozone distributor with the lid and optional protective grille removed.

FIG. 3 represents a top view of an ozone distributor with the lid and optional protective grille removed.

FIG. 4 represents a bottom view of an ozone distributor with the base and optional second protective grille removed.

FIG. 5 represents an ozone generation unit having integral ozone generation plates and energize circuitry.

FIG. 6 represents a larger ozone generation unit having detached ozone generation plates and energize circuitry residing on a dedicated circuit board.

FIG. 7 represents a pivotable ozone distribution unit including ozone distributor rotatably mounted to cart.

### DETAILED DESCRIPTION

A portable, vortex driven ozone distributor is disclosed that includes a vortex fan and multiple ozone generation units within a canister. The vortex fan in combination with the canister provides the ability generate a high velocity stream of ozonated air that may be directed against the ceiling of a room. The high velocity of the ozonated airstream permits all surfaces of the room to be sanitized by the ozone because the air traverses the ceiling and follows the walls down to contact all surfaces of the room.

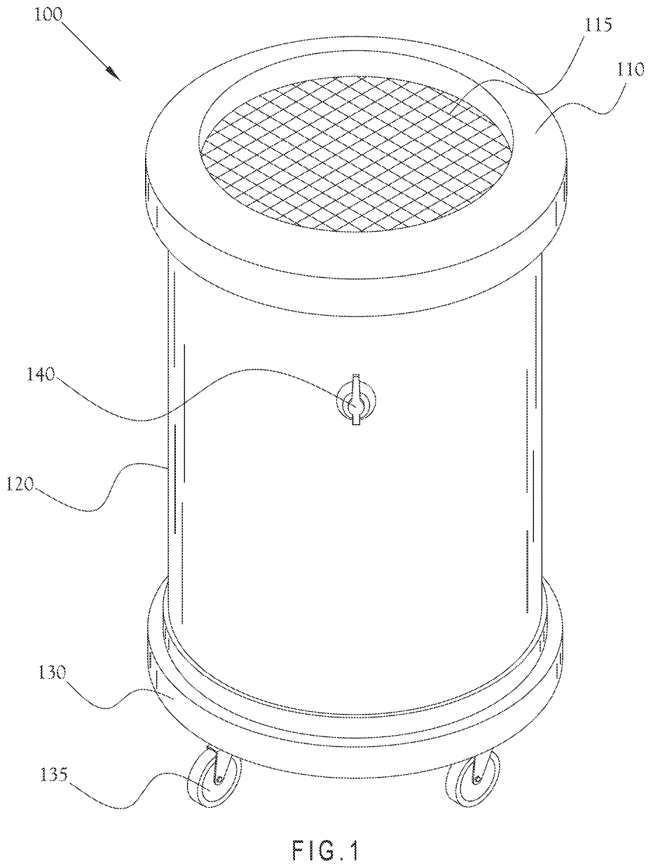

FIG. 1 represents a portable, vortex driven ozone distributor 100. The distributor 100 includes a lid 110, a cannister 120, and a base 130. The lid 110 may optionally be fitted with a protective grille 115 so large debris cannot fall into the unit. The protective grille 115 also may be configured to prevent operator hands and fingers from contacting interior components (not shown) of the distributor 100. The cannister 120 encloses the interior components of the distributor 100 and preferably is cylindrical, however other shapes may be used that are compatible with the enclosed interior components and the desired vortex airflow through the distributor 100.

The base 130 provides support to the cannister 120. The base 130 optionally may be provided with casters for easy portability of the distributor 100 from location to location. While not shown in the figure, the base 130 optionally may be equipped with a second protective grille so large debris are not drawn into the distributor 100 during use. While the lid 110, the canister 120, and the base 130 are generally represented as separate components in the figure, any one or more of these constituents or others may be integrated into another.

In operation, ambient air enters through the base 130, passes through the interior of the cannister 120 that encloses the interior components and exits through the lid 110. Control switch 140 resides on the front of the canister 120 for easy operation of the distributor 100 by the user. The control switch 140 may be located otherwise on the distributor 100 or may be eliminated if the distributor 100 is under wireless control.

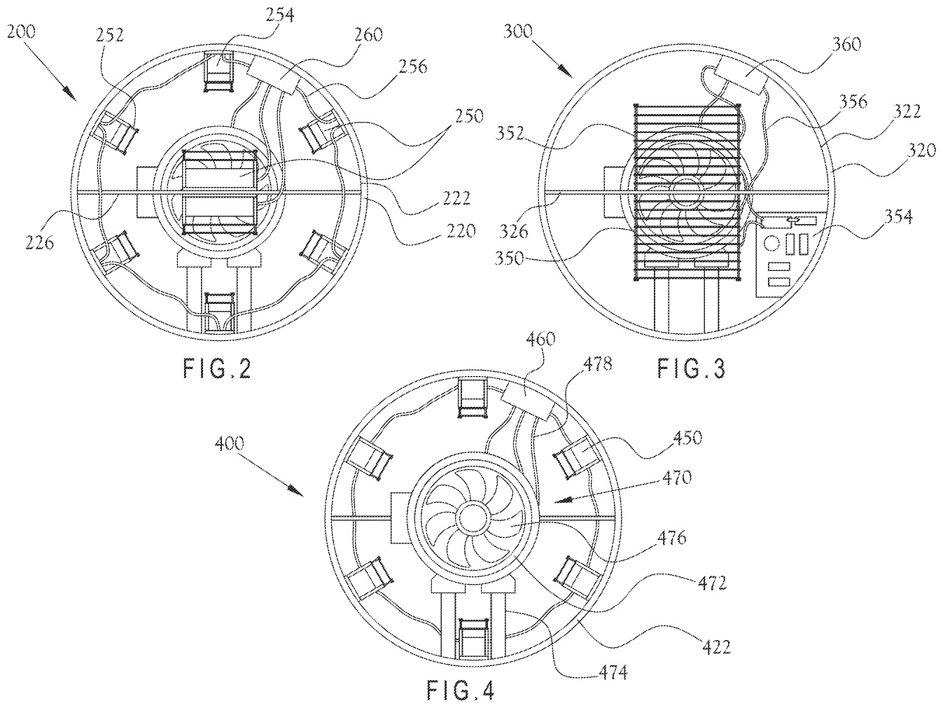

FIG. 2 represents a top view of an ozone distributor 200 with the lid and optional protective grille removed. In this representation, eight ozone generation units 250 are represented, where six units are directly affixed to an interior 222 of a canister 220 and two additional units are affixed to support bar 226 traversing a diameter of the canister 220. The ozone generation units 250 may be otherwise mounted within the canister 220 as to maximize airflow through the plates. While not shown in the figure, preferably, the ozone generation units 250 are affixed to the lid of the ozone distributor 200 so the ozone generation units 250 may be removed for service by removing the lid. The ozone generation units 250 preferably reside in the upper half, more preferably the upper third, and most preferably within about ten centimeters of the exit opening of the canister 220. Thus, the ozone generation units 250 preferably reside near the upper opening of the canister 220 where air is expelled from the canister 200.

Each of the represented units 250 includes both ozone generation plates 252 and energize circuitry 254 required to energize the plates 252 to produce ozone in response to an applied electric potential. Wires 256 connect each of the units 250 to control module 260, which includes control electronics (not shown) to direct an electric potential to one or more of the units 250. Thus, while the wiring may appear to be serial, preferably it is parallel, thus with each unit 250 having independent connection to the control module 260. In this manner the control module 260 can energize one or more of the units 250 simultaneously, or to turn all the units 250 off. Preferably, the distributor 200 includes enough of the units 250 to generate from 200 to 300 grams (g) of ozone per hour in oxygen, more preferably the distributor 200 includes enough of the units 250 to generate from 220 to 290 grams (g) of ozone per hour in oxygen.

The control module 260 may be controlled manually by the user via one or more switches exterior to the interior 222 of the canister 220, remotely through a wireless interface, or thought a combination of manual user input and wireless control. The control module preferably receives signals from an ozone concentration monitor (not shown) positioned within the enclosed space that informs the control module 260 what ozone concentration is being generated within the enclosed space. Preferably the ozone concentration monitor resided within the interior 222 of the canister 220.

FIG. 3 represents a top view of an ozone distributor 300 with the lid and optional protective grille removed. However, unlike in the representation of FIG. 2, in FIG. 3 the eight individual ozone generation units are replaced with two, larger ozone generation units 350. The larger units 350 lack integration of the generation plates and the energize circuitry, instead having plates 352 separated from energize circuitry 354. This design change in the larger ozone generation units 350 is desirable due to the substantially larger energize circuitry required to energize the additional plates. Preferably, the larger ozone generation units produce at least as much ozone per unit time as the smaller plates, and allow individual control of the larger units 350 by control module 360. A similar support bar 326 may be used to position the larger units 350 in the airflow.

FIG. 4 represents a bottom view of an ozone distributor 400 with the base and optional second protective grille removed. Vortex fan 470 is positioned substantially centrally within a diameter of canister interior 422. The fan 470 includes a vortex generating housing 472, housing support brackets 474 that affix the housing 472 to the canister interior 422, and vortex generating fan blade 476. The fan blade 476 spins in response to an applied electrical potential provided through wires 478 in response to a control signal from the control module 460. The combination of the vortex generating housing 472 and the vortex generating fan blade 476 generate a high velocity airstream that maintains a vortex while passing through the canister interior 422 and through ozone generation units 450 before exiting through the lid (not shown).

The vortex fan 470 preferably resides in the lower half, more preferably the lower third, of a longitudinal height of the canister interior 422. Thus, the vortex fan 470 preferably resides near the lower opening of the canister interior 422 where air is drawn into the canister.

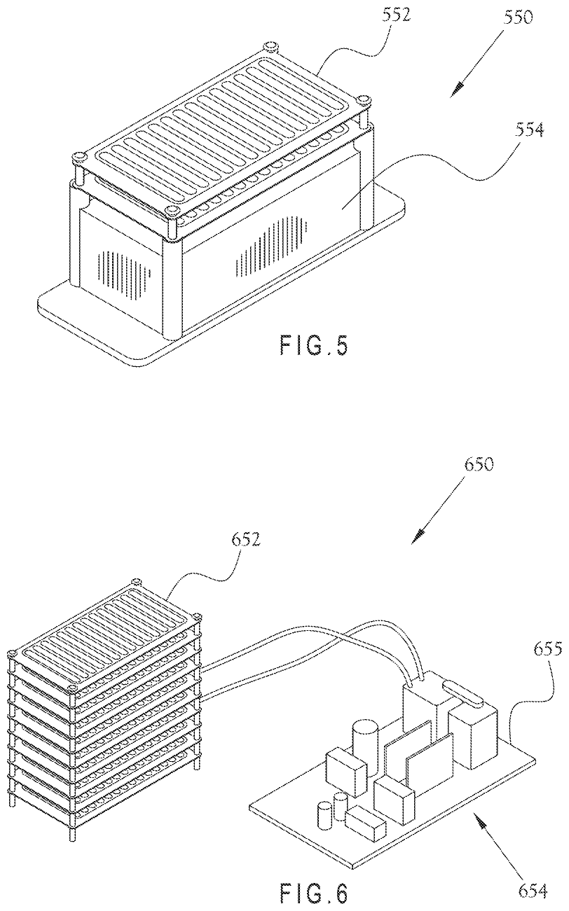

FIG. 5 represents an ozone generation unit 550 having integral ozone generation plates 552 and energize circuitry 554. In the unit 550, the plates 552 include two separate ozone generation plates. Preferable ozone generation units having integral plates can produce approximately 16 grams (g) of ozone per hour in oxygen and are available from SIHONTM, China, for example.

FIG. 6 represents a larger ozone generation unit 650 having detached ozone generation plates 652 and energize circuitry 654 residing on a dedicated circuit board 655. In the unit 650, the plates 652 include ten separate ozone generation plates, thus having the ability to produce approximately five times the amount of ozone per unit time in relation to a two-plate unit. Preferable larger ozone generation units having detached plates can produce approximately 80 grams (g) of ozone per hour in oxygen and are available from SIHON™, China, for example. Preferably, three of the ozone generation units 650 are used in the ozone distribution unit to provide an ozone generation capacity of approximately 240 g of zone per hour in oxygen.

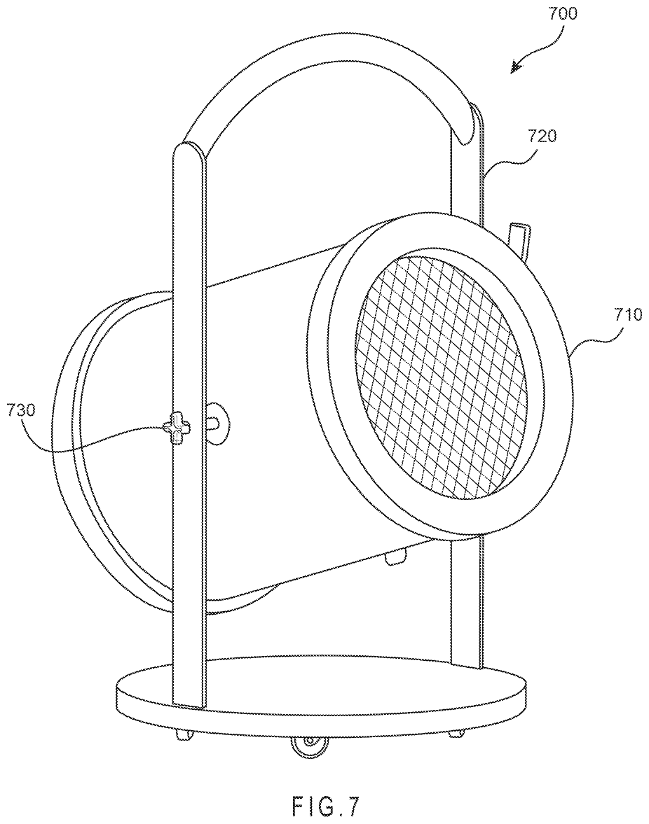

FIG. 7 represents a pivotable ozone distribution unit 700 including ozone distributor 710 rotatably mounted to cart 720. The cart includes rotating connectors 730 that allow the ozone distributor 710 to be rotated from a vertical to an approximately 115-degree down angle from vertical with the base. By rotating the ozone distributor 710 away from vertical, the ozonated air may be directed as desired. For example, the ozone distributor 710 may be rotated to direct ozonated air directly into the return ducting of a heating, ventilating, and air conditioning system or into a basement window, and the like.

The distributor distributes the generated ozone via a substantially vertical air column that rotates—thus being a vortex. The vortex provides the ability of the distributor to generate an ozonated airflow that contacts the ceiling of the enclosed space. When the vortex contacts the ceiling the ozonated air traverses out and follows the ceiling of the room before traversing down the sidewalls of the enclosed space. As the system maintains continuous ozonated airflow across substantially the entire ceiling of the airspace, the ozone, being heaver than air, falls on substantially every surface in the room.

Additionally, the air turbulence created throughout the room further distributes the ozone into nooks and crannies that are otherwise difficult or impossible to reach with a sterilant. The vertical vortex created by the distributor overcomes a significant disadvantage of conventional units that mostly sanitize the floor of the room and raised flat surfaces, such as desks, near the conventional ozone generation unit. As the vortex of ozonated air contacts the ceiling, traverses the ceiling, and traverses down the sidewalls of the enclosed space, ozone sensors positioned at the distributor and at each corner of the room preferably read an ozone concentration of ±5% within five minutes of activating the distributor.

The flow capacity of the vortex fan is determined in relation to the ceiling height of the room to be sanitized. Preferably the vortex fan can produce from 85 to at least 850 cubic-feet-per-minute (CFM). A suitable vortex fan for use in the distributor is the Cloudline S8 8-inch fan as available from AC infinity. However, other vortex fans may be used.

The canister of the distributor may be extended vertically so the output of the distributor is brought closer to the ceiling in taller rooms. Vertical extension of the canister can be provided by an add-on cone, tube, hose, and the like that also may be used to direct the ozonated vortex in a direction other than vertical.

Preferably, the distributor is configured with regard to the ability to produce ozone per unit time so that in relation to the volume of the room to be sanitized the distributor can produce a room ozone concentration of at least two parts-per-million (ppm) within three minutes of activating the distributor. More preferably, the distributor is configured to produce a room ozone concentration of at least three ppm within three minutes of activating the distributor within the room to be sanitized. Preferably, a room may be sanitized by operation of the distributor for at least seven to fifteen minutes within the room, more preferably for at least nine to fifteen minutes.

The distributor may be used to sanitize a multi-room building where the method includes placing a distributor in each of at least two, preferably four rooms, turning on the distributors to sanitize the at least two, preferably four rooms, and after sterilization moving the distributors to at least two, preferably four different rooms. In this way, all the rooms in the building are sanitized consecutively, with a subset of at least two, preferably four rooms being sanitized simultaneously.

To provide a clear and more consistent understanding of the specification and claims of this application, the following definitions are provided.

Vortex refers to a rotating column of air that has a greater velocity than a non-rotating air column generated from the same energy. The rotating column of air of a vortex includes a region in the column of air in which the flow revolves around an axis line. In a vortex, once directional flow is established within a volume of still air, the moving, high(er) speed air will cause the still air to be “pulled” into the stream, as air molecules travelling together experience fewer collisions. The higher pressure from the relatively still air surrounding the vortex is compressing the vortex toward its axis of rotation, forcing it in toward its axis of rotation and maintaining its shape, rather than dissipating as would normally occur without the vortex.

Bacteria includes, but is not limited to, Salmonella enteritidis, E. coli 0157:H7, Listeria monocytogenes, Shigella dysenteriae, Micrococcus aureus, Clostriduim botulinum, Mycobacterium tuberculosis, Bacillus anthracis, and Streptococcus sp.

Mold includes, but is not limited to, Botrytis, Rhizopus, Penicillium, and Aspergillus.

Sanitize means to substantially reduce the context of microorganisms present. Substantially reduce means that at least 70% of the previously living bacteria, viruses, fungus, or mold is killed. Higher kill rates, such as what is referred to as a three (99.9%) to four (99.99%) log reduction or “kill” are preferred. A three-log reduction may be referred to as sanitize or sanitizing, while a four-log reduction may be referred to as disinfect or disinfecting. The terms sterilize and sterilization are often used in the context of a six-log-plus (99.9999+) reduction.

Electrical communication includes at least one of electrically connected and non-electrically connected: where electrically connected means components communicate with each other by means of a conducting path such as through a wire, a cable, other conductors, circuitry, combinations, and the like; and non-electrically connected means components communicate with each other with or without a conducting path such as with radio signals, lasers, cellular or other telephones, WIFI (wireless fidelity) or other wireless network protocols, satellites, combinations, and the like. Components with electrical communication may be both electrically connected and non-electrically connected; for example, components may be electrically connected to supply electrical power and non-electrically connected to transfer data and operating signals. “Electrical communication” also includes when components are operatively connected to perform a particular function.

Unless otherwise indicated, all numbers expressing quantities of ozone used or times in the specification and claims are to be understood as indicating both the exact values as shown and as being modified by the term “about”. Thus, unless indicated to the contrary, the numerical values of the specification and claims are approximations that may vary depending on the desired properties sought to be obtained. At the very least, and not as an attempt to limit the application of the doctrine of equivalents to the scope of the claims, each numerical parameter should at least be construed considering the number of reported significant digits and by applying ordinary rounding techniques.

Unless the context clearly dictates otherwise, where a range of values is provided, each intervening value to the tenth of the unit of the lower limit between the lower limit and the upper limit of the range is included in the range of values.

Spatially relative terms, such as “up,” “down,” “right,” “left,” “beneath,” “below,” “lower,” “above,” “upper” and the like, may be used for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below”, or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The simplified diagrams and drawings do not illustrate all the various connections and assemblies of the various components, however, those skilled in the art will understand how to implement such connections and assemblies, based on the illustrated components, figures, and provided descriptions.

1. A portable, vortex driven ozone distributor, comprising:
a lid connected to a canister that resides on a base, where the canister comprises:
at least two ozone generation units fixed within the canister where the ozone generation units comprise ozone generation plates and energize circuitry;
at least one control module in electrical communication with the at least two ozone generation units configured to instruct the energize circuitry to energize the ozone generation plates with a first applied electric potential, where the at least one control module can instruct one or more of the at least two ozone generation units in response to a control signal; and
a vortex fan in electrical communication with the at least one control module, where the vortex fan comprises a vortex generating housing and fan blade configured to spin in response to a second applied electric potential provided in response to the at least one control module.

2. The distributor of claim 1, where at least six ozone generation units are fixed within the canister.

3. The distributor of claim 1, where the at least two ozone generation units generate from 200 to 300 grams of ozone per hour in oxygen when energized.

4. The distributor of claim 1, where the at least two ozone generation units are affixed to the lid.

5. The distributor of claim 1, where the at least two ozone generation units reside within a top half of the longitudinal height of the canister.

6. The distributor of claim 1, where the vortex fan resides within a bottom half of the longitudinal height of the canister.

7. The distributor of claim 1, where the lid comprises a protective grille.

8. The distributor of claim 1, where the canister is cylindrical.

9. The distributor of claim 1, where a control switch reside on a front face of the canister and provides the control signal.

10. The distributor of claim 1, where the control signal is provided wirelessly.

11. The distributor of claim 2, where the canister further comprises two additional ozone generation units affixed to a support bar traversing a diameter of the canister.

12. The distributor of claim 1, further comprising an ozone concentration monitor configured to provide at least a portion of the control signal.

13. The distributor of claim 1, where the ozone generation units each comprise from two to ten separate ozone generation plates that in combination are configured to produce from 16 to 80 grams of zone per hour in oxygen.

14. The distributor of claim 1, further comprising a cart to which the vortex driven ozone distributor is rotatably mounted.

15. The distributor of claim 1, where the distributor is configured to provide a column of rotating air that contacts a ceiling of an enclosed airspace, traverses across the ceiling of the enclosed airspace, and then traverses down sidewalls of the enclosed airspace.

16. The distributor of claim 1, where the vortex fan produces from 85 to 850 cubic feet per minute of airflow through the canister.

17. The distributor of claim 1, where the distributor achieves an ozone concentration of at least two parts-per-million within an enclosed airspace after activation.

18. A method of using the vortex driven ozone distributor of claim 1 to sanitize multiple enclosed airspaces, such as multiple rooms of a building, the method comprising:
placing at least two of the distributors in at least two enclosed airspaces;
activating the distributors to sanitize the at least two enclosed airspaces;
moving the at least two distributors to at least two different enclosed airspaces; and
activating the distributors to sanitize the at least two different enclosed airspaces.

19. The method of claim 18, where the distributors are activated for at least seven to fifteen minutes within each of the enclosed airspaces and achieve ozone concentrations within each of the enclosed airspaces of 2 to 3 parts-per-million.

20. The method of claim 18, where ozone sensors located at the distributors and at each corner of the enclosed airspaces read ozone concentrations within ±5% within five minutes of activating the distributors.
